(19) United States
(12) Patent Application Publication
(10) Pub. No.: US 2006/0293664 A1
(43) Pub. Date: Dec. 28, 2006

(54) Orthopaedic anchorage element and osteosynthesis device

(76) Inventor: Jorg Schumacher, Tuttlingen (DE)

Correspondence Address: RATNERPRESTIA, P O BOX 980, VALLEY FORGE, PA, 19482-0980, US

(21) Appl. No.: 11/416,564

(22) Filed: May 3, 2006

(30) Foreign Application Priority Data
May 4, 2005 (DE) 10 2005 021 879.2

(51) Int. Cl. A61F 2/30

(52) U.S. Cl. 606/61

(57) ABSTRACT

In order to improve an orthopaedic anchorage element for anchorage in or on at least one bone portion such that the stability thereof is increased, wherein the element comprises an anchorage part for fixing to the at least one bone portion, a seating part including at least one connecting element seating for a connecting element of an osteosynthesis device for connecting two anchorage elements and a clamping screw having a first and a second end of the screw for fixing the connecting element in the at least one connecting element seating, wherein the seating part comprises a seating part thread and the clamping screw comprises a clamping screw thread corresponding to the seating part thread and wherein the clamping screw thread comprises a thread end which merges into the first end of the screw, it is proposed that the first turn of thread of the clamping screw thread be at least partially removed commencing from the end of the thread merging into the first end of the screw. Furthermore, an improved osteosynthesis device is proposed.

The present disclosure relates to the subject matter disclosed in German patent application number 10 2005 021 879.2 of May 4, 2005, which is incorporated herein by reference in its entirety and for all purposes.

### BACKGROUND OF THE INVENTION

The present invention relates to an orthopaedic anchorage element for anchorage in or on at least one bone portion, comprising an anchorage part for fixing to the at least one bone portion, a seating part including at least one connecting element seating for a connecting element of an osteosynthesis device for connecting two anchorage elements and a clamping screw having a first and a second end of the screw for fixing the connecting element in the at least one connecting element seating, wherein the seating part comprises a seating part thread and the clamping screw comprises a clamping screw thread corresponding to the seating part thread, wherein the clamping screw thread comprises a thread end which merges into the first end of the screw.

Furthermore, the present invention relates to an osteosynthesis device for fixing at least two bone portions relative to one another in a fixing position, comprising at least two anchorage elements and a connecting element that is adapted to be fixed to at least two of the at least two anchorage elements.

An orthopaedic anchorage element and an osteosynthesis device of the type described hereinabove are known from US 2004/0049196 A1 for example. The anchorage element is in the form of a bone screw and comprises a clamping screw for fixing a rod to a fork shaped screw head whose clamping screw thread comprises a sharply tapered thread end which merges into the first end of the screw. Thus, especially in the case of undercut threads, there remains a particularly sharp undercut burr at the end of the thread, as is illustrated in exemplary manner in FIG. 11. This leads, especially in the case of undercut threads, to the thickness of the material at the base of the thread, i.e. in the transitional region from the screw core to the threaded profile thereof, being smaller than at the outer diameter of the profile of the clamping screw. This causes an additional notch effect on the flanks of the thread. The disadvantage here is that the load-carrying capacity of the flanks of the thread is decreased with reducing material thickness. This can lead to premature failure of the clamping screw thread due to an application of force to the flanks of the thread in the run-out section of the clamping screw thread and, especially when tightening the screw, this can cause an incipient crack in the base of the thread. In the worst case, there would even be a propagation of the crack under dynamic load and finally the consequence would be a complete failure of the clamping screw thread.

Consequently, it would be desirable to improve an orthopaedic anchorage element and an osteosynthesis device of the type described hereinabove in such a way that the stability thereof is increased.

### SUMMARY OF THE INVENTION

In accordance with the invention, it is advantageous to provide an orthopaedic anchorage element of the type described hereinabove wherein the first turn of thread of the clamping screw thread is at least partially removed commencing from the end of the thread merging into the first end of the screw.

Due to the removal of the sharply tapering end of the thread that merges into the first end of the screw in the known anchorage elements, it is precisely that portion of the clamping screw thread that is removed which is particularly weak due to the reducing thickness of the material in the run-out of the thread. In particular, the clamping screw thread is removed to an extent such that the material thickness of its profile is sufficient to ensure a requisite load-carrying capacity and to withstand an expected load, especially too, a dynamic load. Furthermore, an incipient crack in the base of the thread when tightening the clamping screw can be prevented in this manner. The propagation of a crack and an eventual failure of the clamping screw thread can thereby be entirely discounted. In addition, by virtue of the further development in accordance with the invention of known orthopaedic anchorage elements, the length of the clamping screw or the number of turns of its thread can be minimized. The total overall length of the anchorage element can thereby be minimized. A screw in the real sense of having an external thread or a nut or a screw ring having an internal thread can be used as the clamping screw. It is self-evident that the at least partial removal of the first turn of thread of the clamping screw thread commencing from the end of the thread merging into the first end of the screw is also to be understood as meaning that this part of the clamping screw thread can be omitted during the production process right from the beginning, as would be possible for example by making the clamping screw by means of a casting or sintering process. The crucial thing is that this section of the clamping screw thread is missing. Furthermore, just the second end or else both ends of the clamping screw could also be formed in the manner described, namely in that the first turn of thread of the clamping screw thread is at least partially removed commencing from the end of the thread merging into the second end of the screw or, however, the last turn of thread, or that the first or the last turn of thread or both turns of the thread are at least partially removed.

The production of the anchorage element is particularly simple, if the first turn of thread is at least partially milled away commencing from the beginning of the thread that is to have its run-out at the first end of the screw. It would also be conceivable for the beginning of the thread to be removed by an eroding or grinding process.

In order to improve the stability of the anchorage element still further, it is advantageous if the at least partially removed first turn of thread is removed in such a manner that the remaining part of the first turn of thread comprises a thread-turn end which has a fully formed thread profile in cross section. In this way, it is ensured that each section of the clamping screw thread is formed such as to be fully capable of carrying the load. The danger of a failure of the clamping screw thread in the region of the thread-turn end is therefore neither more nor less great than for another section of the clamping screw thread.

In order to facilitate the insertion of the clamping screw into the seating part thread, it is expedient if the remaining part of the first turn of thread comprising a thread-turn end is at least partially provided with a chamfer commencing from the end of the thread-turn thereof. The chamfer is preferably arranged in such a manner that the thread profile is not weakened, in particular, not at the base of the thread.

Advantageously, the chamfer on the remaining part of the turn of thread is arranged in a direction pointing away from the first and the second end of the screw. This facilitates the insertion of the clamping screw with its clamping screw thread into the seating part thread of the seating part on the one hand, whilst preventing the thread profile from being weakened, especially in the case of an undercut clamping screw thread, on the other.

In order to ensure a maximum possible load-carrying capacity of the clamping screw thread up to the end of the thread-turn, it is expedient if the end of the thread-turn comprises an end face which intersects a radial plane containing a longitudinal axis of the clamping screw. Moreover, the clamping screw is then particularly easy to manufacture.

It is advantageous if the remaining part of the first turn of thread ends with an end face pointing essentially in a direction transverse to a longitudinal axis. The end of the thread-turn is thus somewhat chamfered, this thereby facilitating the insertion of the clamping screw into the seating part thread.

The production of the anchorage element is simplified still further if the end face is curved concavely in a direction away from the end of the thread-turn. Such a shape of end face can be produced in a particularly satisfactory and simple manner using a milling tool.

It is expedient for the first turn of thread of the clamping screw thread to be removed within an angular range of 20° to 150° extending about the screw axis commencing from the end of the thread merging into the first end of the screw.

This means for example that the first 20° of the first turn of thread can be removed, the first 90° or even the first 150°. Depending upon the shape of the thread profile, the effect can thereby be achieved that only sufficiently stable sections of the first turn of thread remain.

In order to prevent the connecting element that is held in clamped manner by the clamping screw in the connecting element seating from being damaged, it is advantageous if the first end of the clamping screw is curved convexly in the direction of the screw axis pointing away from a screw body of the clamping screw.

In order to achieve particularly satisfactory clamping of the connecting element in the connecting element seating, the first end of the clamping screw is preferably formed in the shape of a cone in the direction of the screw axis pointing away from a screw body of the clamping screw. This makes it possible, in particular, for the first end of the clamping screw to rest in an areal or at least linear manner on the connecting element if it is inserted into a connecting element seating that is inclined relative to the screw axis.

In order to minimize the overall length of the anchorage element, it is advantageous if the clamping screw thread comprises two to three turns of thread. In particular, these turns of thread can have a fully formed profile.

It is expedient for the seating part thread to comprise a seating part thread end which runs-out in the direction of the connecting element seating. In this way, there is formed a stop for the clamping screw thread which can thus only be screwed to the seating part to the extent that the seating part thread is fully formed therein. The run-out of the seating part thread thus forms a stop for the clamping screw thread. Over-tightening of the clamping screw is thereby prevented.

For optimal mounting and as large as possible surface-area matching of the connecting element seating to the connecting element that is to be inserted therein, it is advantageous if the connecting element seating comprises a thread-free section which extends in the axial direction for a length that corresponds to a thickness or a diameter of a connecting element, and if the seating part thread adjoins the thread-free section. Damage to the seating part thread by the connecting element or damage to the connecting element by the seating part thread can be prevented by the thread-free section.

Preferably, the seating part thread extends from the thread-free section to a free end of the connecting element seating. This makes it possible for the clamping screw to be brought into engagement with the seating part commencing from the free end of the connecting element seating. In this way, the overall length of the anchorage element can be additionally reduced.

For optimal force transfer and in order to obtain a compact construction of the anchorage element, it is advantageous if a pitch of the clamping screw thread lies in a range of from 0.5 to 2 mm.

In order to achieve a secure connection between the seating part and the clamping screw, it is advantageous if a number of turns of thread of the seating part thread that have a fully formed profile corresponds at least to a number of turns of thread of the clamping screw thread that have a fully formed profile. This makes it possible to connect the clamping screw to the seating part in such a way that all the turns of thread of the clamping screw thread fully engage in the turns of thread of the seating part thread.

The structure of the anchorage element is particularly simple, if the seating part comprises a fork-shaped retaining section incorporating a slot. The fork-shaped retaining section is particularly well suited for the seating of a connecting element. The connecting element can thus be inserted or slid into the slot which forms a seating for the connecting element.

Preferably, the retaining section is provided with the seating part thread. In this way, the clamping screw can be connected to the retaining section and the connecting element fixed to the retaining section.

In accordance with a preferred embodiment of the invention, provision may be made for the connecting element seating to be bounded by two limbs of the fork-shaped retaining section which project in the proximal direction. The structure of the anchorage element is then particularly simple.

It can be advantageous if the seating part thread begins to run-out in one of the two limbs. The terminating, no longer fully formed, seating part thread thus forms a stop for the clamping screw thread of the clamping screw which can no longer engage in the terminating section of the seating part thread. In this way, pre-loading of the screw such as to exceed a permissible maximum and the occurrence of damage to the clamping screw thread and/or the seating part thread can be prevented since a further increase in torque when screwing in the clamping screw does not cause further rotation thereof and thus there is no further increase of the pre-loading. Moreover, over-tightening of the clamping screw is also prevented. The length of the clamping screw or the distance of the end of the thread of the clamping screw thread from the first end of the clamping screw is preferably determined in such a way that the necessary minimum pre-loading for achieving a desired clamping effect is reached before the run-out of the thread reaches the stop formed by the termination of the seating part thread. Self-evidently, it would also be conceivable to provide a stop which is not formed by a termination of the seating part thread, but, for example, by an additional projection on the seating part against which the clamping screw or the clamping screw thread impinges.

Furthermore, it can be advantageous if, in a connecting position of the anchorage element in which a connecting element is held in clamped manner in the connecting element seating, the end of the turns of thread of the clamping screw thread is arranged in the region of the slot between the two limbs. It is thereby ensured that only fully formed thread profile sections or at least thread profile sections of the clamping screw that are of adequate load-bearing capacity engage in the seating part thread of the seating part. Furthermore, if the seating part thread runs out in one of the two limbs, then this limb forms a stop for the end of the turns of thread of the clamping screw thread since it cannot engage any further into a corresponding seating part thread.

In order to ensure sufficient stability of the anchorage element, it is expedient if the slot has a width which defines an opening angle commencing from a longitudinal axis of the retaining section within a range of from 30° to 70°. Furthermore, a maximum width of the connecting element that is capable of being inserted into the connecting element seating is also defined thereby.

For a particularly compact arrangement of the anchorage element, it is expedient if the clamping screw thread is an external thread and the seating part thread is an internal thread.

The stability of the anchorage element can be increased if the clamping screw thread is an internal thread and the seating part thread is an external thread. In particular, by the provision of a fork-shaped retaining section, the clamping screw, which can be constructed in the form of a nut or a screw ring, holds together the two limbs which could be pressed apart as a result of too high a tightening torque.

In order to enable the clamping screw to be screwed to the seating part in a particularly simple manner, the latter part expediently comprises a seating for a tool pointing in the proximal direction.

The structure of the anchorage element is particularly simple, if a screw core of the clamping screw is cylindrical in shape or is formed such as to be substantially cylindrical.

Furthermore, an overall length of the anchorage element can be minimized if an outer diameter of the clamping screw is greater than a length of the clamping screw parallel to the screw axis.

The production of the anchorage element is further simplified if the clamping screw thread is a single flight thread. Furthermore, a relative positioning of the clamping screw thread with respect to the seating part thread in a connecting position in which the connecting element is held in clamped manner in the connecting element seating can also be effected thereby in a particularly simple manner.

Preferably, a profile of the clamping screw thread and/or of the seating part thread is undercut. A particularly good connection between the clamping screw and the seating part can thereby be achieved.

It is expedient for an undercut of the profile to point in the proximal direction. In the case of the clamping screw in accordance with the invention, the proximal direction means in the direction of its second end.

The structure of the anchorage element is particularly simple, if the anchorage part and the seating part are formed in one piece.

In order to enable optimal adjustment of the anchorage element to a direction given by the connecting element and in addition to simplify the production process, the anchorage part and the seating part are advantageously formed such as to be connectable in releasable manner. In particular, the anchorage part and the seating part can be formed such as to be moveable relative to one another. Furthermore, the seating part can be formed, for example, in the form of a head mounted on the anchorage part.

In order to enable a connection of the anchorage element to a bone portion to be effected in a simple manner, it is expedient if the anchorage part comprises a bone thread for screwing the anchorage part into the bone portion.

However, it can also be expedient if the anchorage part is constructed in the form of a bone hook or a bone nail.

The production of the anchorage element is simplified, if the clamping screw thread and the seating part thread are formed coaxially with respect to the longitudinal axis of the seating part.

It is advantageous for a plane of symmetry of the connecting element seating to contain the longitudinal axis of the seating part. This further simplifies the production of the anchorage element.

In accordance with a preferred embodiment of the invention, provision may be made for the seating part to be mounted on the anchorage part, for the seating part to be moveable relative to the anchorage part in an adjusting position and to be fixed immovably to the anchorage part in a connecting position. This arrangement permits the anchorage element to be fixed in or on a bone portion and to subsequently bring the seating part into a desired position in which the connecting element can or should be introduced into the connecting element seating.

Preferably, at least one of the two limbs of the retaining section comprises a predetermined breaking point. The overall length of the anchorage element can thereby be minimized in a desired manner. Thus, it is possible on the one hand to introduce the connecting element into the connecting element seating and secure it by the clamping screw, without however fixing the connecting element to the seating part in clamped manner. Due to the predetermined breaking point, a part of the limb can be removed when the connecting element is fixed in a desired manner. A protruding part of the retaining section can thus be removed in a simple manner.

It is advantageous, if there is provided a clamping body which is mounted on the seating part in moveable manner and if the clamping body is adapted to be clamped by the clamping screw directly or indirectly against the seating part or against a coupling element of the anchorage part. By appropriate choice of the clamping body, manufacturing tolerances can be compensated during the production of the anchorage element. Furthermore, an optimal degree of force can be exerted by the clamping screw directly or indirectly on the connecting element by appropriate shaping of the clamping body.

Particularly effective clamping of the connecting element in the connecting element seating can be achieved if it is adapted to be fixed between the clamping screw and the clamping body in clamped manner.

In accordance with the invention, the object postulated hereinabove is achieved in the case of an osteosynthesis device of the type described hereinabove in that at least one of the at least two anchorage elements of the osteosynthesis device is one of the anchorage elements described above.

As a matter of choice, just particular ones or else all the anchorage elements of the osteosynthesis device can be formed in the manner proposed above in accordance with the invention.

Furthermore the object postulated hereinabove is achieved by a method of producing an orthopaedic anchorage element for anchorage in or on at least one bone portion, comprising an anchorage part for fixing to the at least one bone portion, a seating part including at least one connecting element seating for a connecting element of an osteosynthesis device for connecting two anchorage elements and a clamping screw having a first and a second end of the screw for fixing the connecting element in the at least one connecting element seating, wherein the seating part is provided with a seating part thread and the clamping screw is provided with a clamping screw thread corresponding to the seating part thread, wherein the clamping screw thread is formed in such a manner that it comprises a thread end which merges into the first end of the screw, and wherein the first turn of thread of the clamping screw thread is at least partially removed commencing from the end of the thread merging into the first end of the screw.

The production process in accordance with the invention enables the clamping screw to be produced in a conventional manner and only further modified as required i.e. to remove a part of the end of the thread.

The method is particularly easy to carry out if the first turn of thread is at least partially milled away commencing from the beginning of the thread which is to run-out at the first end of the screw. Alternatively, the beginning of the thread could also be ground away, eroded away or etched away.

### BRIEF DESCRIPTION OF THE DRAWINGS

The following description of preferred embodiments of the invention will serve for a more detailed explanation taken in conjunction with the drawing. Therein:

FIG. 1: shows a perspective illustration of a bone screw with a first embodiment of a clamping screw;

FIG. 2: a perspective view of the bone screw in FIG. 1 including limbs of the screw head that are separated at a predetermined breaking point;

FIG. 3: an enlarged view of the region A in FIG. 2;

FIG. 4: a longitudinal sectional view through the portion depicted in FIG. 3;

FIG. 5: a perspective view of the clamping screw for the anchorage element in FIG. 1;

FIG. 6: a view of the clamping screw in FIG. 5 in the direction of its first end;

FIG. 7: a perspective view of a second embodiment of a clamping screw;

FIG. 8: a view of the clamping screw in FIG. 7 in the direction of its first end;

FIG. 9: a perspective view of a third embodiment of a clamping screw;

FIG. 10: a view of the clamping screw in FIG. 9 in the direction of its first end;

FIG. 11: a perspective view of a clamping screw known from the state of the art;

FIG. 12: a view of the bone screw in FIG. 11 in the direction of its first end; and

FIG. 13: a side view of an osteosynthesis device in accordance with the invention.

### DETAILED DESCRIPTION OF THE DRAWINGS

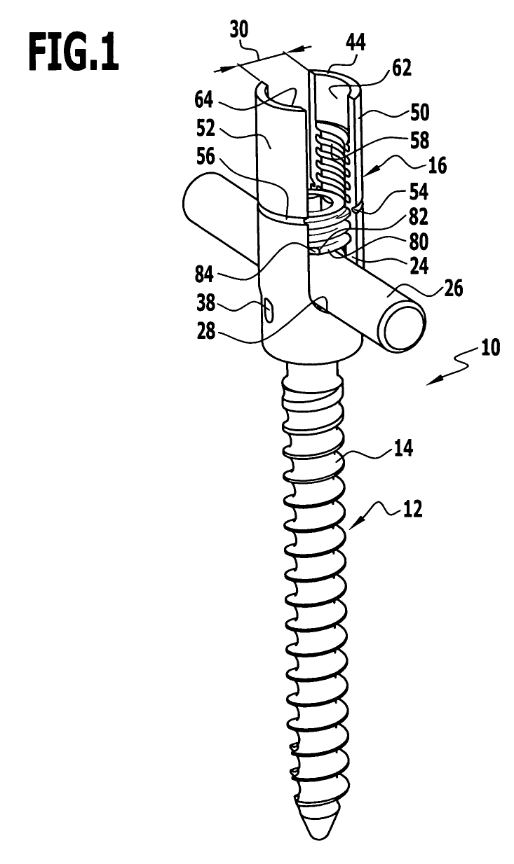

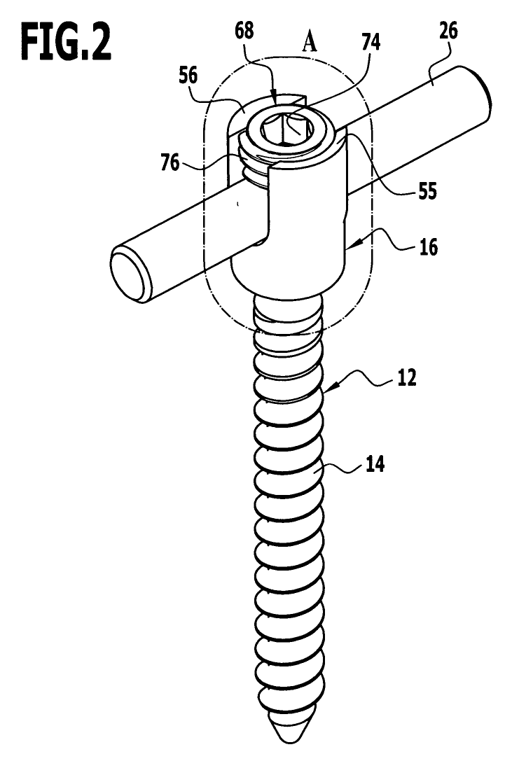

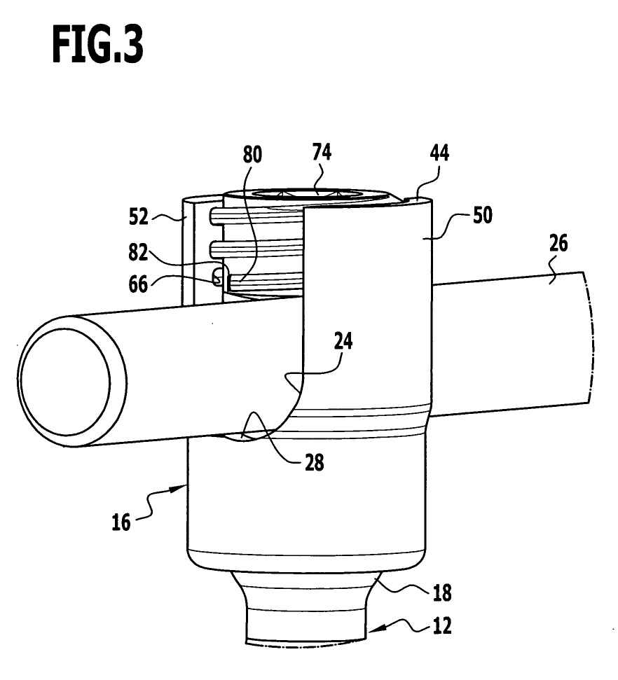

A first exemplary embodiment of an anchorage element in accordance with the invention in the form of a pedicle screw 10 is illustrated in FIGS. 1 to 4. The pedicle screw 10 is constructed in the form of a poly-axial screw, i.e. an anchorage part in the form of a screw body 12 that is provided with a bone thread 14 is connected by a ball joint connection to a seating part in the form of a fork head 16. For this purpose, a proximal end of the screw body 12 is provided with a spherical head 18 which can be seated in a hollow dome shaped seat 20 at a distal end 22 of the fork head 16.

The fork head 16 is substantially sleeve-shaped and comprises a slot 24 which serves as a connecting element seating for the insertion of a connecting element, for example, in the form of a rod 26 or of a plate-like element having a rod-shaped section. The slot 24 has the shape of a semicircle at the end 28 thereof pointing towards the seat 20 and this is matched to an outer diameter of the rod 26. In like manner, the width 30 of the slot 24 corresponds approximately to the width of the rod 26.

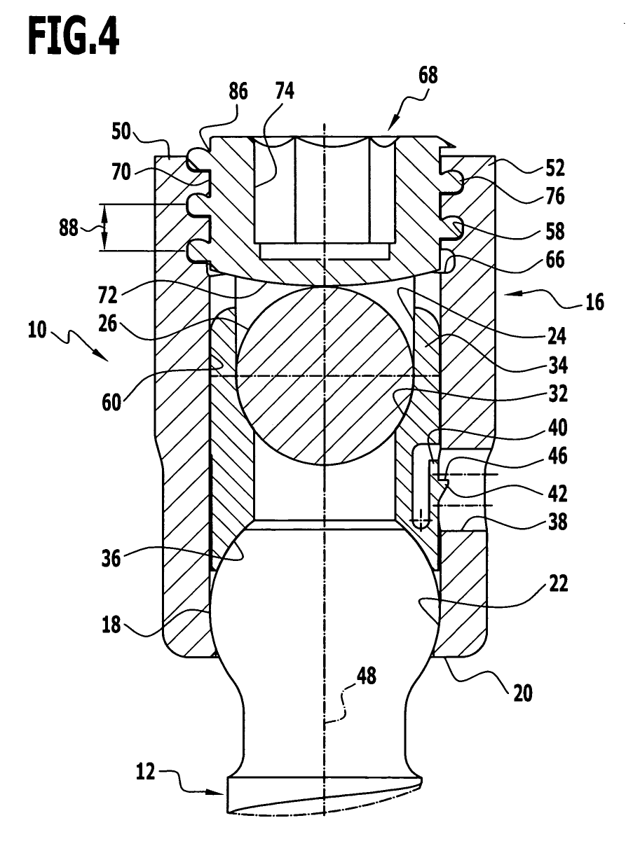

The rod 26 does not rest directly on the lower or distal end 28 of the slot 24, but rather, on a seat 32 of a clamping body 34 which is of a substantially hollow cylindrical shape, said seat being matched to an outer contour of the rod 26 and comprising seating surfaces of hollow cylindrical shape. An end pointing towards the head 18 and resting thereon is provided with an edge 36 of hollow dome-shaped form which is directly adjacent the head 18. In order to keep the clamping body 34 captive on the fork head 16, a small break-through 38 is provided in the fork head adjacent to the seat 20, a latching nose 42 that protrudes from a spring tongue 40 projecting into said break-through when the clamping body 34 is pushed into the fork head 16 such as to come from a proximal end 44 thereof. During such an insertion process, the latching nose 42 glides on an inner wall of the fork head 16, whereby the spring tongue 40 is pressed radially inward in the direction of an axis of symmetry 48 of the fork head 16. The latching tongue 40 springs back into its starting position illustrated in FIG. 4 as soon as the latching nose 42 can enter the break-through 38. A latching surface 46 of the latching nose 42 points in the proximal direction and thereby prevents the clamping body 34 from falling out of the fork head 16 in the proximal direction.

The fork head 16 comprises two mutually diametrically opposite limbs 50 and 52 which are separated by the slot 24. Both of the limbs 50 and 52 are provided with a respective groove 54 and 56 which is located on an outer surface and forms a predetermined breaking point in order to remove a proximal part of the limbs 50 and 52 after the rod 26 has been fixed to the fork head 16.

In the interior of the fork head 16, there is provided an internal thread 58 which adjoins a thread-free inner wall section 60 that extends in the proximal direction commencing from the seat 20. Both of the limbs 50 and 52 comprise a respective further thread-free wall section 62 and 64 which extends between the internal thread 58 and the end 44. The internal thread 58 is formed in such a manner that it runs-out in the limb 52. A terminating last turn of thread is provided with the reference symbol 66 in FIG. 4.

For the purposes of fixing the rod 26, there serves a clamping screw 68 which comprises a cylindrical screw body 70 whose first end 72 pointing in the distal direction is curved convexly in a direction pointing away from the screw body 70. A proximal end of the screw body 70 is provided with a seating for a tool in the form of a female hexagon 74 which is coaxial with respect to the axis of symmetry 48. Instead of the female hexagon seating 74, provision could also be made for any other type of internal polygonal seating or even an internal polygonal seating having rounded edges, for example in the form of a Torx® seating.

Furthermore, the screw body 70 is provided with an external thread 76 which is formed in correspondence with the internal thread 58. It comprises approximately two fully formed turns of thread and runs-out at the proximal end of the clamping screw 68. This terminating end of the thread is additionally provided with a chamfer 78 which points essentially in the proximal direction.

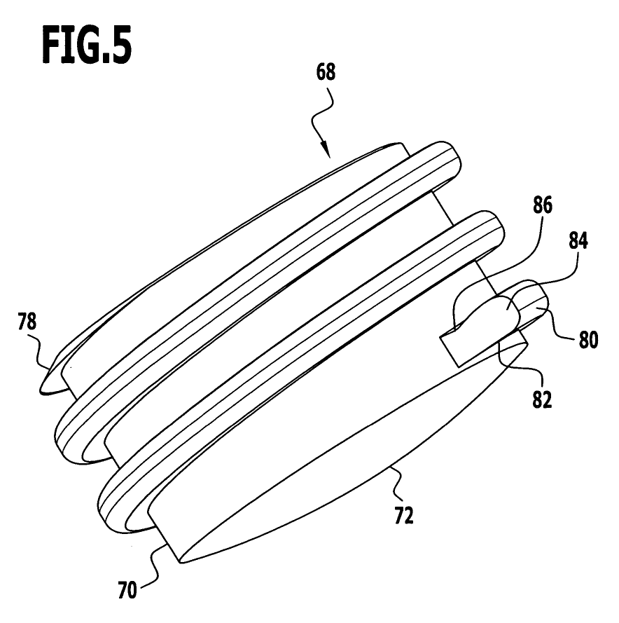

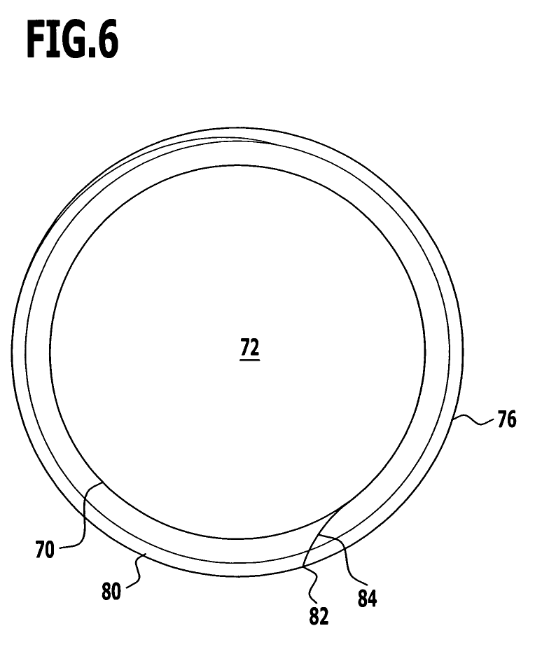

A first turn of thread of the external thread 76 is partially removed commencing from an end of the thread merging into the end 72, namely, as can be perceived particularly well in FIGS. 5 and 6, to an extent such that the remaining part of the first turn of thread 80 comprises a thread-turn end 82 which has a fully formed thread profile in cross section. The end of the turn of thread 82 comprises an end face 84 which is curved concavely in a direction pointing away from the end of the turn of thread 82. The external thread 76 is undercut, i.e. it comprises an undercut 84 which can be perceived particularly well in FIGS. 4 and 5

In order to fix the rod 26 to the fork head 16, the clamping screw 68 is inserted into the end 44 in a direction coming from the proximal, and the thread-turn end 82 of the external thread 76 thereof engages in the internal thread 58. The clamping screw 68 can be screwed into the fork head 16 until such time as the rod 26 is held in clamped manner between the clamping body 34 and the clamping screw 68. At the same time, the force applied via the rod 26 to the clamping body 34 is also used for pressing the head against the seat 20 whereby the fork head 16 is fixed to the head 18 of the screw body 12 in the connecting position illustrated in FIGS. 1 to 4.

The dimensions of the clamping screw 68 are selected in such a way that the end of the turn of thread 82 is arranged in the region of the slot 24 between the limbs 50 and 52. A stop for the end of the turn of thread 82 is formed in the limb 52 by the terminating turn of thread 66 of the internal thread 58. In consequence, the clamping screw 68 no longer finds an “entrance” for the end of the turn of thread 82 in the limb 52. This thus prevents the pre-loading of the screw exceeding a permissible maximum and also prevents the occurrence of damage to the thread. A limit on the torque used for tightening the arrangement is thus provided. Furthermore, over-tightening of the clamping screw is prevented in the manner described. It is optimal for the length of the clamping screw 68 or that of the internal thread 58 to be matched to a diameter of the rod 26. A range of tolerance is defined by the width 30 of the slot 24 or by an opening angle of the slot 24 in the circumferential direction which amounts to approximately 50°, but could also lie in a range of from 30° to 70° in the case of the exemplary embodiment of the pedicle screw illustrated in FIGS. 1 to 4. A pitch 88 of the external thread 76 amounts to approximately 1.5 mm.

As is not difficult to appreciate in FIGS. 1 to 6, somewhat more than two fully formed turns of thread are provided on the clamping screw 68.

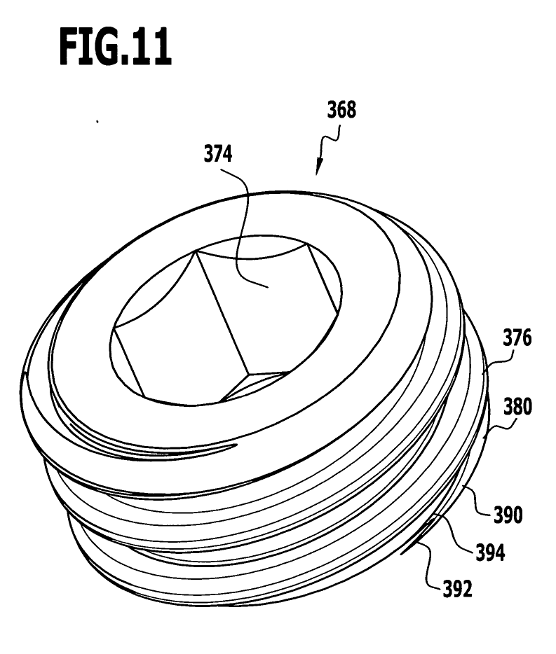

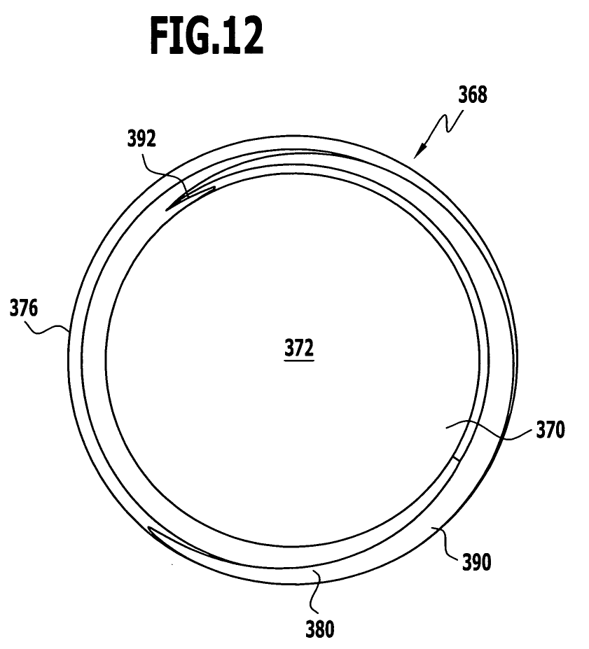

If one compares the clamping screw 68 with a clamping screw 368 that is illustrated in FIGS. 11 and 12 and is known from the state of the art, then it will be noticed that a first turn of thread 380 merges into the distal end 372 of the screw body 370. Furthermore, since the external thread 376 is provided with a chamfer 390, there remains a fine burr 392 which projects at the end of the turn of thread due to the undercut profile of the external thread 376. Moreover, as can readily be perceived in FIG. 11, a base of the thread 394 is significantly weakened in the direction of the burr 392, whereby a full load cannot be applied to a first part of the turn of thread 380. Consequently, in use of the clamping screw 368 and in the event of excessive tightening, an incipient crack can occur in the base of the thread 394 whereby propagation of the crack under dynamic load is possible, something which can finally entail failure of the entire external thread 376. The clamping screw 68 does not exhibit this disadvantage since the end of the turn of thread 82 ends with a fully formed thread profile.

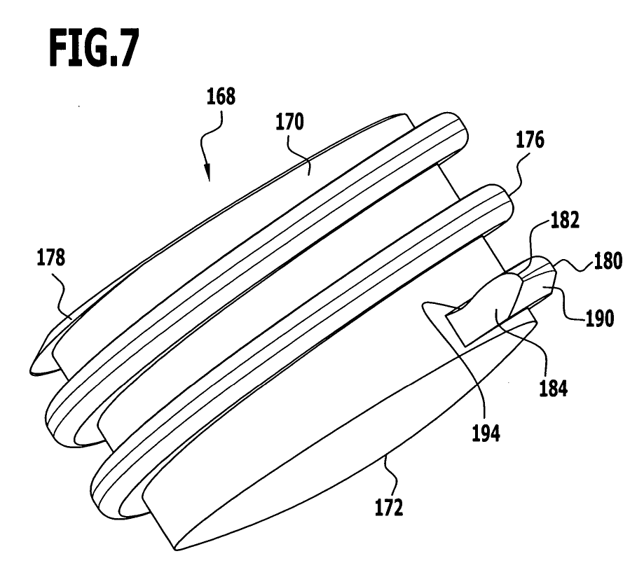

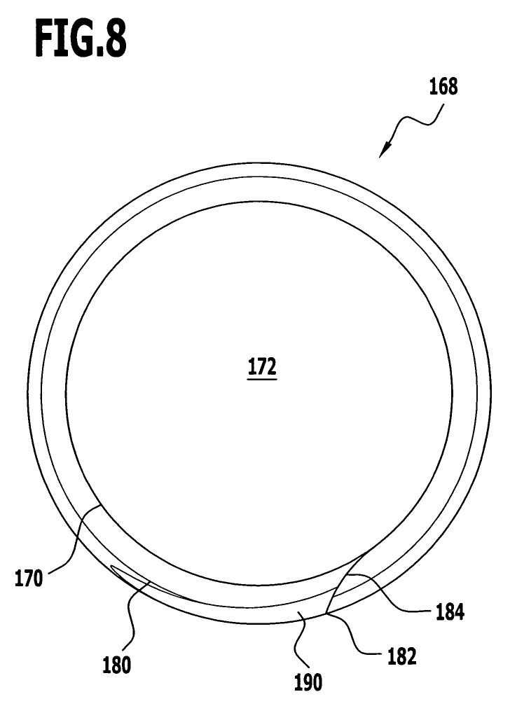

A second embodiment of a clamping screw bearing the general reference symbol 168 is provided in FIGS. 7 and 8. The clamping screw 168 is formed almost identically to the clamping screw 68. The only difference is a chamfer 190 which is provided in the vicinity of the first turn of thread 180 and points essentially in the distal direction and which facilitates somewhat the introduction of the external thread 76 into the internal thread 58. The chamfer 190 is provided in such a way that a remaining profile of the external thread 176 does not exhibit a weakening in the base of the thread 194 especially in the region of the end of the turn of thread 182 and concomitantly in the region of the end face 184. The chamfer 190 can extend approximately over an angular range of from 40° to 180°.

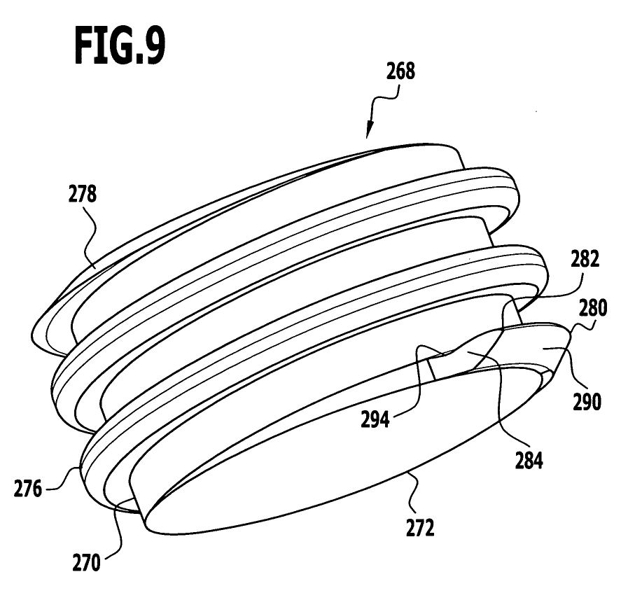

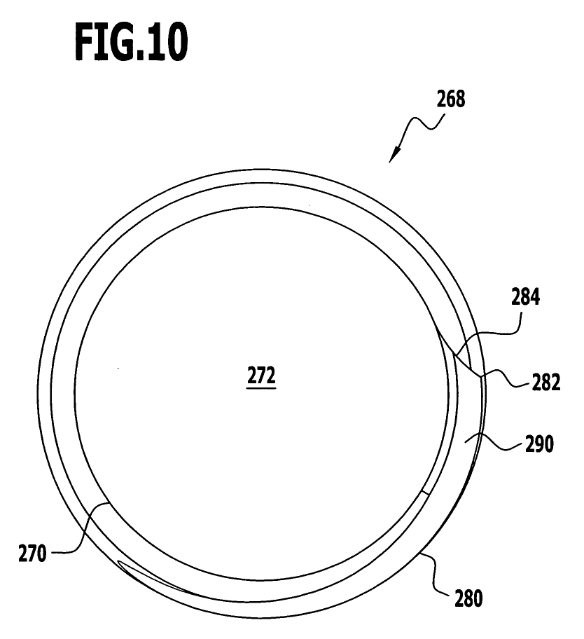

A third embodiment of a clamping screw bearing the general reference symbol 268 is provided in FIGS. 9 and 10. The first turn of thread 280, which merges into the end 372 in the case of the clamping screw 368, is partially removed in the case of the clamping screw 268, namely, commencing from an end of the external thread 276 that merges into the end 272. There thus remains an end face 284 on the end of the turn of thread 282 which is somewhat smaller than the end face 84 of the clamping screw 68, however, the base of the thread of the clamping screw 268 is not weakened so that even with the provision of a chamfer 290 that is formed in analogy with the chamfer 190 and points essentially in the distal direction, the external thread 276 is not weakened not even in the remaining part of the first turn of thread 280.

In the case of the clamping screws 68, 168 and 268, the first part of the first turn of thread is removed by milling. Alternatively, this part of the external thread could also be removed by an eroding or grinding process.

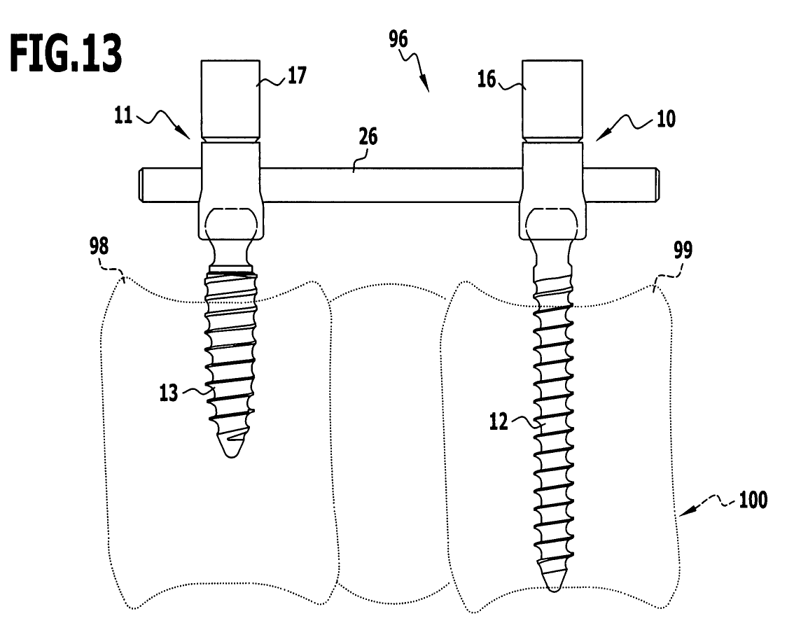

An osteosynthesis device bearing the general reference symbol 96 is shown in FIG. 13, this device comprising in exemplary manner two pedicle screws 10 and 11 as well as a rod 26. The pedicle screws 10 and 11 are screwed into vertebral bodies 98 and 99 of a spinal column 100 for the purposes of fixing the rod 26 and enable the rod to be fixed to their fork heads 16 and 17. The pedicle screws 10 and 11 are constructed in the form of poly-axial screws, i.e. the fork heads 16 and 17 are moveable relative to the screw bodies 12 and 13 in an adjusting position. The two vertebral bodies 98 and 99 or else two bone portions can be held in a desired position relative to one another with the osteosynthesis device 96 for example.

What is claimed is:

1. An orthopaedic anchorage element for anchorage in or on at least one bone portion, comprising an anchorage part for fixing to the at least one bone portion, a seating part including at least one connecting element seating for a connecting element of an osteosynthesis device for connecting two anchorage elements and a clamping screw having a first and a second end of the screw for fixing the connecting element in the at least one connecting element seating, wherein the seating part comprises a seating part thread and the clamping screw comprises a clamping screw thread corresponding to the seating part thread, wherein the clamping screw thread comprises a thread end which merges into the first end of the screw, wherein the first turn of thread of the clamping screw thread is at least partially removed commencing from the end of the thread merging into the first end of the screw.

2. An anchorage element in accordance with claim 1, wherein the first turn of thread is at least partially milled away commencing from the beginning of the thread that is to have its run-out at the first end of the screw.

3. An anchorage element in accordance with claim 1, wherein the at least partially removed first turn of thread is removed in such a manner that the remaining part of the first turn of thread comprises a thread-turn end which has a fully formed thread profile in cross section.

4. An anchorage element in accordance with claim 1, wherein the remaining part of the first turn of thread comprising a thread-turn end is at least partially provided with a chamfer commencing from its thread-turn end.

5. An anchorage element in accordance with claim 4, wherein the chamfer on the remaining part of the turn of thread is arranged in a direction pointing away from the first and the second end of the screw.

6. An anchorage element in accordance with claim 3, wherein the thread-turn end comprises an end face which intersects a radial plane containing a longitudinal axis of the clamping screw.

7. An anchorage element in accordance with claim 1, wherein the remaining part of the first turn of thread ends with an end face pointing substantially in a direction transverse to a longitudinal axis.

8. An anchorage element in accordance with claim 6, wherein the end face is curved concavely in a direction pointing away from the thread-turn end.

9. An anchorage element in accordance with claim 1, wherein the first turn of thread of the clamping screw thread is removed in an angular range extending about the screw axis of 20° to 150° commencing from the end of the thread merging into the first end of the screw.

10. An anchorage element in accordance with claim 1, wherein the first end of the clamping screw is curved convexly in the direction of the screw axis pointing away from a screw body of the clamping screw.

11. An anchorage element in accordance with claim 1, wherein the first end of the clamping screw is formed in the shape of a cone in the direction of the screw axis pointing away from a screw body of the clamping screw.

12. An anchorage element in accordance with claim 1, wherein the clamping screw thread comprises two to three turns of thread.

13. An anchorage element in accordance with claim 1, wherein the seating part thread comprises a seating part thread end terminating in the direction of the connecting element seating.

14. An anchorage element in accordance with claim 1, wherein the connecting element seating comprises a thread-free section that extends in the axial direction for a length which corresponds approximately to a thickness or a diameter of a connecting element, and the seating part thread adjoins the thread-free section.

15. An anchorage element in accordance with claim 14, wherein the seating part thread extends from the thread-free section up to a free end of the connecting element seating.

16. An anchorage element in accordance with claim 1, wherein a pitch of the clamping screw thread lies in a range of from 0.5 to 2 mm.

17. An anchorage element in accordance with claim 1, wherein a number of turns of thread of the seating part thread that have a fully formed profile corresponds at least to a number of turns of thread of the clamping screw thread that have a fully formed profile.

18. An anchorage element in accordance with claim 1, wherein the seating part comprises a fork-shaped retaining section incorporating a slot.

19. An anchorage element in accordance with claim 18, wherein the retaining section is provided with the seating part thread.

20. An anchorage element in accordance with claim 18, wherein the connecting element seating is bounded by two limbs of the fork-shaped retaining section which project in the proximal direction.

21. An anchorage element in accordance with claim 20, wherein the seating part thread begins to run-out in one of the two limbs.

22. An anchorage element in accordance with claim 18, wherein in a connecting position of the anchorage element in which a connecting element is held in the connecting element seating in clamped manner, the end of the turns of thread of the clamping screw thread is arranged in the region of the slot between the two limbs.

23. An anchorage element in accordance with claim 1, wherein the slot has a width which defines an opening angle commencing from a longitudinal axis of the retaining section in a range of from 30° to 70°.

24. An anchorage element in accordance with claim 1, wherein the clamping screw thread is an external thread and the seating part thread is an internal thread.

25. An anchorage element in accordance with claim 1, wherein the clamping screw thread is an internal thread and the seating part thread is an external thread.

26. An anchorage element in accordance with claim 1, wherein the clamping screw comprises a tool seating pointing in the proximal direction.

27. An anchorage element in accordance with claim 1, wherein a screw core of the clamping screw is cylindrical or is substantially cylindrical.

28. An anchorage element in accordance with claim 1, wherein an outer diameter of the clamping screw is greater than a length of the clamping screw parallel to the screw axis.

29. An anchorage element in accordance with claim 1, wherein the clamping screw thread is a single flight thread.

30. An anchorage element in accordance with claim 1, wherein a profile of the clamping screw thread and/or of the seating part thread is undercut.

31. An anchorage element in accordance with claim 30, wherein an undercut of the profile points in the proximal direction.

32. An anchorage element in accordance with claim 1, wherein the anchorage part and the seating part are formed in one piece.

33. An anchorage element in accordance with claim 1, wherein the anchorage part and the seating part are connectable in releasable manner.

34. An anchorage element in accordance with claim 1, wherein the anchorage part comprises a bone thread for screwing the anchorage part into a bone portion.

35. An anchorage element in accordance with claim 1, wherein the anchorage part is in the form of a bone hook or a bone nail.

36. An anchorage element in accordance with claim 1, wherein the clamping screw thread and the seating part thread are formed coaxially with respect to a longitudinal axis of the seating part.

37. An anchorage element in accordance with claim 1, wherein a plane of symmetry of the connecting element seating contains the longitudinal axis of the seating part.

38. An anchorage element in accordance with claim 37, wherein the seating part is mounted on the anchorage part, and the seating part is moveable relative to the anchorage part in an adjusting position and is immovably fixed to the anchorage part in a connecting position.

39. An anchorage element in accordance with claim 20, wherein at least one of the two limbs of the retaining section comprises a predetermined breaking point.

40. An anchorage element in accordance with claim 1, wherein there is provided a clamping body which is mounted in moveable manner on the seating part, and the clamping body is adapted to be clamped by the clamping screw directly or indirectly against the seating part or against a coupling element of the anchorage part.

41. An anchorage element in accordance with claim 40, wherein the connecting element is adapted to be fixed between the clamping screw and the clamping body in clamped manner.

42. An osteosynthesis device for fixing at least two bone portions relative to one another in a fixing position, comprising at least two anchorage elements and a connecting element that is adapted to be fixed to at least two of the at least two anchorage elements, wherein at least one of the at least two anchorage elements of the osteosynthesis device is an anchorage element comprising an anchorage part for fixing to the at least one bone portion, a seating part including at least one connecting element seating for a connecting element of an osteosynthesis device for connecting two anchorage elements and a clamping screw having a first and a second end of the screw for fixing the connecting element in the at least one connecting element seating, wherein the seating part comprises a seating part thread and the clamping screw comprises a clamping screw thread corresponding to the seating part thread, wherein the clamping screw thread comprises a thread end which merges into the first end of the screw, wherein the first turn of thread of the clamping screw thread is at least partially removed commencing from the end of the thread merging into the first end of the screw.

43. A method of producing an orthopaedic anchorage element for anchorage in or on at least one bone portion, comprising an anchorage part for fixing to the at least one bone portion, a seating part including at least one connecting element seating for a connecting element of an osteosynthesis device for connecting two anchorage elements and a clamping screw having a first and a second end of the screw for fixing the connecting element in the at least one connecting element seating, wherein the seating part is provided with a seating part thread and the clamping screw is provided with a clamping screw thread corresponding to the seating part thread, wherein the clamping screw thread is formed in such a manner that it comprises a thread end which merges into the first end of the screw, wherein the first turn of thread of the clamping screw thread is at least partially removed commencing from the end of the thread merging into the first end of the screw.

44. A method in accordance with claim 43, wherein the first turn of thread is at least partially milled away commencing from the beginning of the thread which is to run-out at the first end of the screw.
